(19) United States
(12) Patent Application Publication
(10) Pub. No.: US 2008/0261866 A1
(43) Pub. Date: Oct. 23, 2008

(54) Compositions Comprising Glycosaminoglycan and Nonsteroidal Anti-Inflammatory Drug

(75) Inventors: Rajesh Jain, New Delhi (IN); Kour Chand Jindal, New Delhi (IN); Sukhjeet Singh, New Delhi (IN)

Correspondence Address: LADAS & PARRY LLP, 26 WEST 61ST STREET, NEW YORK, NY, 10023, US

(73) Assignee: PANACEA BIOTEC LTD., New Delhi (IN)

(21) Appl. No.: 10/586,544

(22) PCT Filed: Jan. 19, 2005
(86) PCT No.: PCT/IN2005/000026

§ 371 Date: Jun. 26, 2008

(51) Int. Cl. A61K 38/00; A61K 31/715; A61P 1/00

(52) U.S. Cl. 514/8; 514/54

(57) ABSTRACT

Pharmaceutical compositions comprising of glycosaminoglycan or salts thereof, preferably chondroitin or salts thereof, more preferably chondroitin sulphate, and nonsteroidal anti-inflammatory drug(s) or salts thereof, optionally with pharmaceutically acceptable excipient(s) are described. The compositions of the present invention provide gastrosparing effect in conditions where nonsteroidal anti-inflammatory drug(s) or their salts are used, particularly in mammals. Also provided are process for the manufacture of such novel compositions and method to minimize the nonsteroidal anti-inflammatory drug(s) induced gastric toxicity.

### FIELD OF THE INVENTION

The present invention relates to novel pharmaceutical compositions comprising of glycosaminoglycan or salts thereof, preferably chondroitin or salts thereof, more preferably chondroitin sulphate, and nonsteroidal anti-inflammatory drug(s) or salts thereof, optionally with pharmaceutically acceptable excipient(s). The compositions of the present invention provide gastrosparing effect in conditions where nonsteroidal anti-inflammatory drug(s) or their salts are used, particularly in mammals. The present invention also provides process for the manufacture of such novel compositions and method to minimize the nonsteroidal anti-inflammatory drug(s) induced gastric toxicity.

### BACKGROUND OF THE INVENTION

Glycosaminoglycans are a type of long, unbranched polysaccharide molecule. They are major structural components of cartilage and are also found in the cornea of the eye. Examples of common glycosaminoglycans include keratan sulphate, chondroitin sulphate, dermatan sulphate, heparin sulphate, and hyaluronan.

Chondroitin sulphate is a sulphated glycosaminoglycan that consists of repeating chains of glycosaminoglycan molecules. It is a sulphated linear polysaccharide constructed of two or three kinds of monosaccharides namely N-acetylgalactosamine, L-iduronic acid, and D-glucuronic acid. It is present in various connective tissues, including cartilage, skin, blood vessels, and bone (Hardingham and Fosang, 1992). It is the major constituent of articular cartilage proteoglycan and plays an important role in the elasticity and function of articular cartilage (Hardingham and Bayliss, 1992). It has been used clinically as a chondroprotective agent for treatment of osteoarthritis (Morreale et al., 1996; Bucsi and Poor, 1998). Chondroitin sulphate is currently marketed and therapeutically prescribed for neurodynia, lumbago and arthrodynia as a cartilage protective agent.

Hori et al. demonstrated the therapeutic efficacy of chondroitin sulphate in protection of digestive mucosa against animal model of inflammatory bowel disease (Hori et al., 2001).

Nonsteroidal anti-inflammatory drugs (NSAIDs) are widely used in the management of pain and inflammation. They are among the most commonly prescribed drugs in the world but their therapeutic effects are limited by their untoward effects on the gastrointestinal tract (Kulkarni and Varghese, 1998; Kulkarni et al., 2000). NSAIDs act by non-selectively inhibiting cyclooxygenase (COX) enzyme thereby blocking prostaglandins release. Two isoforms of COX have been identified namely COX-1 and COX-2. COX-1 is constitutive and involves in house keeping functions such as mucus secretion and renal blood flow; whereas COX-2 is inducible by pain and inflammatory stimulus (Kulkami et al., 2000). The classical NSAIDs inhibit both COX isoforms thereby blocking the useful gastric prostaglandins released by gastric COX-1 leading to gastrointestinal side effects ranging from dyspepsia to life threatening gastrointestinal bleeding and potentially perforating gastro-duodenal ulcers (Garcia Rodriguez and Jick, 1994; Langman et al., 1994).

Various attempts have been made to develop NSAIDs with reduced gastrointestinal side effects which has led to the development of several types of NSAIDs such as selective inhibitors of COX-2 (Brideau et al., 1999), nitric oxide releasing NSAIDs (Takeuchi et al., 1998), NSAIDs pre-associated with zwitter ionic phospholipids (Wallace and Chin, 1997), and NSAIDs complexation with divalent metal ions (Dendrinu-Samara et al., 1998). Of these attempts, the main approach has been to develop safer NSAIDs on the basis of the COX-1 hypothesis.

There still exists an unmet need to develop new combination products or new methods to minimize the NSAID(s) induced gastric toxicity. No composition has been reported in the art where chondroitin or salts thereof, especially chondroitin sulphate is employed in combination with NSAID(s) to minimize the NSAID(s) induced gastric toxicity. The present invention provides novel pharmaceutical compositions comprising of glycosaminoglycan or salts thereof, such as chondroitin sulphate and nonsteroidal anti-inflammatory drug(s).

### OBJECTIVE OF THE INVENTION

It is an objective of the present invention to provide a novel pharmaceutical composition comprising of glycosaminoglycan or salts thereof and at least one nonsteroidal anti-inflammatory drug or salts thereof optionally with pharmaceutically acceptable excipients, wherein the said composition provides a gastrosparing effect and minimizes the gastric toxicity induced by the administration of nonsteroidal anti-inflammatory drug.

It is an objective of the present invention to provide a novel pharmaceutical composition comprising of glycosaminoglycan or salts thereof, preferably chondroitin or salts thereof, more preferably chondroitin sulphate, and at least one nonsteroidal anti-inflammatory drug or salts thereof optionally with pharmaceutically acceptable excipients, wherein the said composition provides a gastrosparing effect and minimizes the gastric toxicity induced by the administration of nonsteroidal anti-inflammatory drug.

It is another objective of the invention to provide a process for preparing such pharmaceutical composition which comprises mixing of glycosaminoglycan or salts thereof and at least one nonsteroidal anti-inflammatory drug or salts thereof optionally with pharmaceutically acceptable excipients and formulating into a suitable dosage form.

It is a further objective of the present invention to provide a method of providing a gastrosparing effect and minimizing the gastric toxicity induced by the administration of nonsteroidal anti-inflammatory drug by administering a pharmaceutical composition comprising of glycosaminoglycan or salts thereof and at least one nonsteroidal anti-inflammatory drug or salts thereof optionally with pharmaceutically acceptable excipients.

### DETAILED DESCRIPTION OF THE INVENTION

The present invention provides novel pharmaceutical composition comprising of glycosaminoglycan or salts thereof and at least one nonsteroidal anti-inflammatory drug or salts thereof optionally with pharmaceutically acceptable excipients. The composition provides a gastrosparing effect and minimizes the gastric toxicity induced by the administration of nonsteroidal anti-inflammatory drug.

In an embodiment, the invention provides novel pharmaceutical compositions comprising of chondroitin or salts thereof, preferably chondroitin sulphate, and nonsteroidal anti-inflammatory drug(s) or salts thereof, optionally with pharmaceutically acceptable excipient(s).

In an embodiment of the present invention, the ratio of glycosaminoglycan or salts thereof and nonsteroidal anti-inflammatory drug or salts thereof in the composition is from 100:1 to 1:100.

In an embodiment of the present invention, the pharmaceutically acceptable excipients are selected from a group comprising but not limited to diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, chelating agents and the like; used either alone or in combination thereof.

The pharmaceutical compositions of the present invention may be administered in the form of conventional pharmaceutical compositions, and may be formulated by means known in the art. The compositions of the present invention can be formulated as oral dosage forms such as tablets, pills, capsules, gels, finely divided powders, dispersions, suspensions, solutions, emulsions, etc; pulmonary and nasal dosage form such as sprays, aerosols, etc.; topical dosage forms such as gels, ointments, creams, etc; parenteral dosage forms; controlled release formulations; fast melt formulations, lyophilized formulations, delayed release formulations, sustained release, extended release formulations, pulsatile release formulations, and mixed immediate release and controlled release formulations. In an embodiment, the composition of the present invention is provided to be taken orally by way of a pediatric suspension, capsule, or tab let.

In another embodiment, the present invention also provides method to minimize the nonsteroidal anti-inflammatory drug(s) induced gastric toxicity. In an embodiment of the present invention, the compositions comprising of glycosaminoglycan or salts thereof and nonsteroidal anti-inflammatory drug(s) or salts thereof is intended to reduce the incidence of dyspepsia, heartburn, bleeding ulcers, and other gastrointestinal complications associated with the use of nonsteroidal anti-inflammatory drug(s).

In yet another embodiment, the present invention also provides process for the manufacture of analgesic and/or anti-inflammatory compositions comprising glycosaminoglycan, preferably chondroitin sulphate or salts thereof and nonsteroidal anti-inflammatory drug(s) or salts thereof. The process for preparing such pharmaceutical composition comprises mixing of glycosaminoglycan or salts thereof and at least one nonsteroidal anti-inflammatory drug or salts thereof optionally with pharmaceutically acceptable excipients and formulating into a suitable dosage form.

The novel pharmaceutical compositions of the present invention are also intended to ensure greater patient compliance and a promising potential to reduce the gastric toxicity associated with use of nonsteroidal anti-inflammatory drug(s). Furthermore, the novel pharmaceutical compositions of present invention are not expected to alter the pharmacodynamic profile of either glycosaminoglycan or nonsteroidal anti-inflammatory drug(s).

In an embodiment, the nonsteroidal anti-inflammatory drug(s) of the present invention includes but is not limited to indomethacin, flurbiprofen, naproxen, diclofenac, ketorolac, mefenamic acid, ibuprofen, ketoprofen, meloxicam, piroxicam, nimesulide, celecoxib, rofecoxib, etoricoxib, parecoxib, valdecoxib, lumiracoxib, licofelone, and the like, or salts thereof.

Examples of common glycosaminoglycans that may be used include keratan sulphate, chondroitin sulphate, dermatan sulphate, heparin sulphate, and hyaluronan.

By careful experimentation, the inventors have found that among the nonsteroidal anti-inflammatory drugs (NSAIDs), Indomethacin and Diclofenac produced severe gastric damage in the stomach. In the present invention, it was surprisingly found that combining Chondroitin sulphate with Indomethacin or Diclofenac significantly attenuated Indomethacin or Diclofenac per se induced gastric ulcerations and perforations in rats; thus producing unexpected gastric protection by minimizing the NSAID induced gastric toxicity.

### Determination of Biological Activity

### NSAID-Induced Acute Gastric Ulcers in Rats

The gastroprotective effect of Chondroitin sulphate was studied on NSAID-induced acute gastric ulcers in rat. The NSAIDs used for the study are Indomethacin and Diclofenac free acid.

### Indomethacin-Induced Acute Gastric Ulcers in Rats

The observed unexpected gastroprotective effect of Chondroitin sulphate is evidenced by test conducted in rats. Wistar rats of either sex were procured from Central Animal House facility, Panacea Biotec Ltd., India. The animals were fasted overnight and those weighing between 150-170 gms at the time of testing were used throughout. All animals were dosed sequentially by the oral route with 0.5% carboxy methylcellulose (CMC) suspension of Indomethacin and/or solutions of Chondroitin sulphate in distilled water. A dosing volume of 10 ml/kg was used for each sequential solution or suspension.

The ulcers were induced as described by Chan et. al. (1995). In brief, overnight fasted animals were administered orally with 20 mg/kg of Indomethacin on the day of experimentation. Chondroitin sulphate was administered at a dose of 50 or 100 mg/kg, 15 minutes before Indomethacin administration. The animals were sacrificed after 4 hours of Indomethacin administration; the stomach was opened along the greater curvature and washed under running water. The number of ulcers was identified using 10× magnifying lens, counted, multiplied by respective score, and summed up for each animal. The mean score as Ulcer index in each group was calculated and compared with Indomethacin group.

Ulcer scoring:

| Size (longest diameter) | Score |
|---|---|
| Ulcers <1 mm | 1 |
| Ulcers 1-3 mm | 5 |
| Ulcers >3 mm | 10 |

The results of the study are presented in Table 1.

TABLE 1
Effect of Chondroitin sulphate on Indomethacin-induced gastric ulcers in
rats

| S. No. | Treatment (mg/kg, p.o.) | Ulcer Index |
|---|---|---|
| 1 | Vehicle control (0.5% CMC) | 2.25 ± 0.48 |
| 2 | Indomethacin control (20) | 66.25 ± 15.8^{a} |
| 3 | Chondroitin sulphate (50) + Indomethacin (20) | 23.0 ± 8.04* |
| 4 | Chondroitin sulphate (100) + Indomethacin | 6.0 ± 3.07* |
| | (20) | |

All the values are expressed as mean ± S.E.M. (Standard Error of Mean).
n (no. of rats) = 6-9 in each treatment group;
*p < 0.05 as compared to control (vehicle) (t-test);
^{a}p < 0.05 as compared to per se treatments (ANOVA followed by Dunnett's test).
Single oral administration of indomethacin 20 mg/kg produced severe gastric damage (red colored, bleeding ulcers and disruption of mucus layer) in fasted animals with ulcer score of 66.25 ± 15.8 when compared to the control value of 2.25 ± 0.48. Oral co-administration of Chondroitin Sulphate 50 mg/kg or 100 mg/kg with Indomethacin 20 mg/kg showed unexpected gastroprotection with ulcer scores of 23.0 ± 8.04 and 6.0 ± 3.07, respectively. Also the extent of gastroprotection was dose dependent as evident from the ulcer index values for 50 and 100 mg/kg of Chondroitin sulphate administered together with 20 mg/kg of Indomethacin (Table 1).

### Diclofenac-Induced Acute Gastric Ulcers in Rats

Wistar rats of either sex were fasted overnight and weighing 170-200 g at the time of testing were used throughout. All animals were dosed sequentially by the oral route with 0.5% carboxy methylcellulose suspension of diclofenac free acid and/or solutions of chondroitin sulphate in distilled water. A dosing volume of 10 ml/kg was used for each sequential solution or suspension. All doses were coded and the test was performed under a code not known to the observer.

The ulcers were induced as described by Chan et al. (1995). In brief, overnight fasted animals were weighed and randomly divided into four groups. Three groups of animals were administered orally with 100 mg/kg of diclofenac free acid and the last group received equivalent volume of vehicle alone on the day of experimentation. Two groups of animals received chondroitin sulphate at a dose of 50 or 100 mg/kg 15 min before diclofenac free acid administration. The animals were sacrificed 4 h after diclofenac free acid administration; the stomach was opened along the greater curvature, and washed under running water. The number of ulcers was identified using 10× magnifying lens, counted, multiplied by respective score, and summed up for each animal. The mean score as ulcer index in each group was calculated as mentioned below.

Ulcer scoring:

| Size (longest diameter) | Score |
|---|---|
| Ulcers <1 mm | 1 |
| Ulcers 1-3 mm | 5 |
| Ulcers >3 mm | 10 |

The results of the study are presented in Table 2.

TABLE 2
Effect of chondroitin sulphate on diclofenac-induced gastric ulcers in rats

| S. No. | Treatment (mg/kg, p.o.) | Ulcer Index |
|---|---|---|
| 1 | Vehicle control (0.5% CMC) | 1.0 ± 0.63 |
| 2 | Diclofenac control (100) | 57.50 ± 11.30^{a} |
| 3 | Chondroitin sulphate (50) + Diclofenac (100) | 19.80 ± 5.52* |
| 4 | Chondroitin sulphate (100) + Diclofenac | 12.00 ± 4.71* |
| | (100) | |

All the values are expressed as mean ± SEM.
n = 5 to 7 in each treatment;
^{a}p < 0.05 as compared to vehicle control;
*p < 0.05 as compared to diclofenac control.

Single oral administration of diclofenac free acid 100 mg/kg produced severe gastric damage (red colored, bleeding ulcers and disruption of mucus layer) in fasted animals when compared to the control animals (Table 1). Oral administration of chondroitin sulphate 50 mg/kg or 100 mg/kg 15 min before the administration of diclofenac free acid 100 mg/kg showed gastroprotection as evidenced by significant reduction in ulcer index as compared to diclofenac free acid control (Table 2).

The examples given below serve to illustrate embodiments of the present invention. However they do not intend to limit the scope of present invention.

### EXAMPLES

Example 1 (Capsule)

| Ingredient | mg/capsule |
|---|---|
| Chondroitin sulphate | 200.0 |
| Indomethacin | 100.0 |
| Microcrystalline cellulose | 200.8 |
| Mannitol | 72.0 |
| Talc | 3.2 |
| Sodium starch glycollate | 12.0 |
| Colloidal silicon dioxide | 12.0 |

### Procedure:

1) Chondroitin sulphate, indomethacin, microcrystalline cellulose and mannitol are sifted and mixed together.
2) Talc, sodium starch glycollate and colloidal silicon dioxide are passed through fine sieves individually and then mixed together.
3) The materials of step 1 and 2 are mixed and filled into empty hard gelatin capsules

Example 2 (Uncoated tablet)

| Ingredient | mg/tablet |
|---|---|
| Chondroitin sulphate | 200.0 |
| Diclofenac free acid | 50.0 |
| Microcrystalline cellulose | 120.0 |
| Mannitol | 80.0 |
| Croscarmellose sodium | 10.0 |
| Lactose | 66.0 |
| Talc | 4.0 |
| Colloidal silicon dioxide | 10.0 |
| Croscarmellose sodium | 10.0 |

### Procedure:

1) Chondroitin sulphate, diclofenac free acid, microcrystalline cellulose, mannitol, croscarmellose sodium and lactose are sifted and mixed together.
2) The material of step 1 is compacted.
3) The compacts of step 2 are passed through sieve and mixed.
4) Talc, colloidal silicon dioxide and croscarmellose sodium are passed through fine sieve and mixed together.
5) The material of step 3 is mixed with material of step 4.
6) The material of step 5 is compressed into tablets.

Example 3 (Uncoated tablet)

| Ingredient | mg/tablet |
|---|---|
| Chondroitin sulphate | 400.0 |
| Nimesulide | 100.0 |
| Microcrystalline cellulose | 120.0 |
| Mannitol | 80.0 |
| Croscarmellose sodium | 10.0 |
| Lactose | 66.0 |
| Talc | 4.0 |
| Colloidal silicon dioxide | 10.0 |
| Croscarmellose sodium | 10.0 |

### Procedure:

1) Chondroitin sulphate, nimesulide, microcrystalline cellulose, mannitol, croscarmellose sodium and lactose are sifted and mixed together.
2) The material of step 1 is compacted.
3) The compacts of step 2 are passed through sieve and mixed.
4) Talc, colloidal silicon dioxide and croscarmellose sodium are passed through fine sieve and mixed together.
5) The material of step 3 is mixed with material of step 4.
6) The material of step 5 is compressed into tablets.

Example 4 (Uncoated tablet)

| Ingredient | mg/tablet |
|---|---|
| Dermatan sulphate | 100.0 |
| Diclofenac sodium | 75.0 |
| Microcrystalline cellulose | 120.0 |
| Mannitol | 80.0 |
| Croscarmellose sodium | 10.0 |
| Lactose | 66.0 |
| Talc | 4.0 |
| Colloidal silicon dioxide | 10.0 |
| Croscarmellose sodium | 10.0 |

### Procedure:

1) Dermatan sulphate, diclofenac sodium, microcrystalline cellulose, mannitol, croscarmellose sodium and lactose are sifted and mixed together.
2) The material of step 1 is compacted.
3) The compacts of step 2 are passed through sieve and mixed.
4) Talc, colloidal silicon dioxide and croscarmellose sodium are passed through fine sieve and mixed together.
5) The material of step 3 is mixed with material of step 4.
6) The material of step 5 is compressed into tablets.

Example 5 (Uncoated tablet)

| Ingredient | mg/tablet |
|---|---|
| Chondroitin sulphate | 200.0 |
| Nimesulide | 100.0 |
| Microcrystalline cellulose | 120.0 |
| Mannitol | 80.0 |
| Croscarmellose sodium | 10.0 |
| Lactose | 66.0 |
| Talc | 4.0 |
| Colloidal silicon dioxide | 10.0 |
| Croscarmellose sodium | 10.0 |

### Procedure:

1) Chondroitin sulphate, nimesulide, microcrystalline cellulose, mannitol, croscarmellose sodium and lactose are sifted and mixed together.
2) The material of step 1 is compacted.
3) The compacts of step 2 are passed through sieve and mixed.
4) Talc, colloidal silicon dioxide and croscarmellose sodium are passed through fine sieve and mixed together.
5) The material of step 3 is mixed with material of step 4.
6) The material of step 5 is compressed into tablets.

Example 6 (Film-coated tablet)

| Ingredient | mg/tablet |
|---|---|
| Core tablet composition | |
| Chondroitin sulphate | 200.0 |
| Diclofenac sodium | 75.0 |
| Microcrystalline cellulose | 120.0 |
| Mannitol | 80.0 |
| Croscarmellose sodium | 10.0 |
| Lactose | 66.0 |
| Talc | 4.0 |
| Colloidal silicon dioxide | 10.0 |
| Croscarmellose sodium | 10.0 |
| Film coating composition | |
| Hydroxypropyl methylcellulose | 12.0 |
| (E-15) | |
| Polyethylene glycol 400 (PEG 400) | 2.4 |
| Iron oxide red | 0.75 |
| Iron oxide yellow | 0.50 |
| Titanium dioxide | 0.25 |
| Isopropyl alcohol | q.s. (lost in processing) |
| Dichloromethane | q.s. (lost in processing) |

### Procedure:

1) Chondroitin sulphate, diclofenac sodium, microcrystalline cellulose, mannitol, croscarmellose sodium and lactose are sifted and mixed together.
2) The material of step 1 is compacted.
3) The compacts of step 2 are passed through sieve and mixed.
4) Talc, colloidal silicon dioxide and croscarmellose sodium are passed through fine sieve and mixed together.
5) The material of step 3 is mixed with material of step 4.
6) The material of step 5 is compressed into tablets.
7) Hydroxypropyl methylcellulose is dispersed in a mixture of isopropyl alcohol and dichloromethane with continuous mixing in homogenizer.
8) PEG 400 is added to the above solution of step 7 and mixed.
9) Iron oxide red, iron oxide yellow and titanium dioxide are passed through fine sieve and mixed.
10) The material of step 9 is added to material of step 8 and mixed for 30 minutes.
11) The core tablets are charged into the coating pan and coated with the coating solution of step 10 till an average tablet weight gain of ˜2-3% is achieved.

1-14. (canceled)

15. A pharmaceutical composition comprising a glycosaminoglycan or salt thereof and at least one nonsteroidal anti-inflammatory drug or salt thereof optionally with pharmaceutically acceptable excipients, wherein the composition provides a gastrosparing effect and minimizes the gastric toxicity induced by the administration of nonsteroidal anti-inflammatory drug.

16. The composition according to claim 15, wherein the glycosaminoglycan is Chondroitin sulphate.

17. The composition according to claim 15, wherein the nonsteroidal anti-inflammatory drug is selected from the group consisting of Indomethacin, flurbiprofen, naproxen, diclofenac, ketorolac, mefenamic acid, ibuprofen, ketoprofen, meloxicam, piroxicam, nimesulide, celecoxib, rofecoxib, etoricoxib, parecoxib, valdecoxib, lumiracoxib, and salts thereof.

18. The composition according to claim 16, wherein the nonsteroidal anti-inflammatory drug is selected from the group consisting of Indomethacin, flurbiprofen, naproxen, diclofenac, ketorolac, mefenamic acid, ibuprofen, ketoprofen, meloxicam, piroxicam, nimesulide, celecoxib, rofecoxib, etoricoxib, parecoxib, valdecoxib, lumiracoxib, and salts thereof.

19. The composition according to claim 15, wherein the ratio of glycosaminoglycan or salt thereof and nonsteroidal anti-inflammatory drug or salt thereof is from 100:1 to 1:100.

20. The composition according to claim 15, wherein the pharmaceutically acceptable excipients are selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents; or a mixture thereof.

21. The composition according to claim 16, wherein the pharmaceutically acceptable excipients are selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents; or a mixture thereof.

22. The composition according to claim 1, which is formulated as an oral; pulmonary, nasal; topical; parenteral; controlled release; fast melt lyophilized; delayed release; sustained release; extended release; pulsatile release; mixed immediate release, or controlled release dosage form.

23. The composition according to claim 22, wherein the oral dosage form is selected from the group consisting of tablets, pills, capsules, gels, finely divided powders, dispersions, suspensions, solutions, and emulsions.

24. The composition according to claim 22, wherein the pulmonary or nasal dosage form is a spray or aerosol.

25. The composition according to claim 22, wherein the topical dosage form is selected from the group consisting of gels, ointments and creams.

26. A process for preparing a pharmaceutical composition according to claim 1, which comprises mixing glycosaminoglycan or salt thereof and at least one nonsteroidal anti-inflammatory drug or salt thereof, optionally with pharmaceutically acceptable excipients and formulating into a dosage form.

27. The process according to claim 26, wherein the glycosaminoglycan is Chondroitin sulphate.

28. A method of providing a gastrosparing effect and minimizing the gastric toxicity induced by the administration of nonsteroidal anti-inflammatory drug by administering a pharmaceutical composition comprising glycosaminoglycan or salt thereof and at least one nonsteroidal anti-inflammatory drug or salt thereof optionally with pharmaceutically acceptable excipients.
